(19) United States
(12) Patent Application Publication
(10) Pub. No.: US 2021/0093293 A1
(43) Pub. Date: Apr. 1, 2021

(54) IMAGING DEVICE AND IMAGING METHOD THEREOF

(71) Applicant: GE Precision Healthcare LLC, Wauwatosa, WI (US)

(72) Inventors: Bing LI, Jiangsu (CN); Qiang YAO, Jiangsu (CN); Liping CHEN, Jiangsu (CN); Lanping LIU, Jiangsu (CN); Kejian SHI, Jiangsu (CN); Lu JIN, Jiangsu (CN)

(21) Appl. No.: 17/023,953

(22) Filed: Sep. 17, 2020

(30) Foreign Application Priority Data
Sep. 30, 2019 (CN) 201910939087.1

(51) Int. Cl. A61B 8/00; F16M 11/28

(52) U.S. Cl. CPC A61B 8/4218; F16H 25/20; F16M 11/28

(57) ABSTRACT

The present invention provides an imaging device, comprising: an adjustable arm; an imaging assembly connected to one end of the adjustable arm; and a counterweight connected to the other end of the adjustable arm through a cable, wherein the counterweight is provided with an actuator, and the actuator is connected to one end of the cable and is capable of driving the cable to move. A method for imaging using an imaging device is further provided, the method comprising: adjusting a position of an imaging assembly; adjusting the length of a cable by using an actuator, so as to adjust an effective weight of the imaging assembly acting on tissue to be imaged; and performing imaging by using the imaging assembly.

### TECHNICAL FIELD

The subject matter disclosed in the present invention relates to the field of medical imaging, and particularly, to an imaging device and an imaging method thereof.

### BACKGROUND ART

Medical imaging devices have wide application in the field of medical diagnosis. Common medical imaging devices include ultrasound imaging devices, magnetic resonance imaging devices, X-ray imaging devices, and the like. For example, a breast ultrasound scanning device is an ultrasound imaging device generating breast images by using echo signals of high-frequency sound waves emitted by a detector in an imaging assembly. In breast examination, breast ultrasound scanning can be used as an auxiliary means for breast cancer screening and is more advantageous for patients having dense breast tissue (for example, a high content of fibro-glandular tissue) than X-ray mammography.

In an example, the breast ultrasound scanning device may be used to image breast tissue in one or a plurality of planes. Before initiating a scan, a user of a scanning device places an imaging assembly on tissue of a scan subject and applies a downward force to the imaging assembly that presses the tissue, so that the tissue can be correctly imaged. The adjustment of the position of the imaging assembly and the adjustment of the force applied downward by the imaging assembly have a significant impact on imaging quality.

### SUMMARY OF INVENTION

Provided in some embodiments of the present invention is an imaging device, comprising: an adjustable arm; an imaging assembly connected to one end of the adjustable arm; and a counterweight connected to the other end of the adjustable arm through a cable, wherein the counterweight is provided with an actuator, and the actuator is connected to one end of the cable and is capable of driving the cable to move.

Optionally, the device further comprises a frame, wherein the frame comprises a guide rail, and the guide rail is configured to guide the counterweight and/or the adjustable arm during movement.

Optionally, the guide rail is a hollow structure having an inner portion in contact with the adjustable arm and an outer portion in contact with the counterweight.

Optionally, the adjustable arm comprises a position-limiting structure for limiting a position of the adjustable arm during movement.

Optionally, portions of the guide rail in contact with the adjustable arm are all non-cylindrical structures.

Optionally, a portion of the adjustable arm in contact with the guide rail is rotatably connected to the remaining portion of the adjustable arm.

Optionally, the other end of the cable is fixedly connected to the adjustable arm.

Optionally, one end of the cable is slidably connected to a bottom of the adjustable arm, and the other end thereof is connected to the counterweight.

Optionally, the bottom of the adjustable arm is provided with a pulley and a blocking plate at least partially surrounding the pulley, a spacing between the pulley and the blocking plate is less than a diameter of the cable, and the cable is slidably connected to the bottom of the adjustable arm through the pulley.

Optionally, the actuator comprises a lead screw motor and a sliding block connected to the lead screw motor, and the sliding block is connected to one end of the cable.

Optionally, the device further comprises a frame and a locking device, wherein the locking device is configured to fix a position of the counterweight.

Optionally, the locking device comprises:

a through-hole, the through-hole being provided in the counterweight at a position close to the frame;

a pin, the pin being at least partially disposed in the through-hole;

a first connecting rod and a pressing rod, one end of the first connecting rod being movably connected to the pin, and the other end thereof being movably connected to the pressing rod;

a second actuator, the second actuator driving the pressing rod to move; and

at least one recess, the recess being provided on the frame and matching the pin.

Optionally, the imaging assembly comprises an ultrasonic transducer.

Also provided in some embodiments of the present invention is a method for imaging using any imaging device described above, the method comprising: adjusting a position of an imaging assembly; adjusting the length of a cable by using an actuator, so as to adjust an effective weight of the imaging assembly acting on tissue to be imaged; and performing imaging by using the imaging assembly.

Optionally, the method further comprises fixing a position of a counterweight by using a locking device.

It should be understood that the brief description above is provided to introduce in simplified form some concepts that will be further described in the Detailed Description of the Embodiments. The brief description above is not meant to identify key or essential features of the claimed subject matter. The protection scope is defined uniquely by the claims that follow the detailed description. Furthermore, the claimed subject matter is not limited to implementations that solve any disadvantages noted above or in any section of the present disclosure.

### BRIEF DESCRIPTION OF THE DRAWINGS

These and other features, aspects, and advantages of the present invention will be better understood when the following detailed description is read with reference to the accompanying drawings, where the same symbols in the drawings represent the same parts throughout the drawings, in which:

FIG. 1 is a schematic structural view of a breast ultrasound scanning device according to some embodiments of the present invention;

FIG. 2 is a schematic structural view of an adjustable arm according to some embodiments of the present invention;

FIG. 3 is a schematic structural view of an adjustable arm according to some other embodiments of the present invention;

FIG. 4 is a bottom view of a guide rail structure according to some embodiments of the present invention;

FIG. 5 is a bottom view of a guide rail structure according to some other embodiments of the present invention;

FIG. 6 is a schematic structural view of a portion of the adjustable arm in contact with the guide rail according to some embodiments of the present invention;

FIG. 7 is a schematic structural view of a counterweight according to some embodiments of the present invention;

FIG. 8 is a schematic structural view of a locking device according to some embodiments of the present invention; and

FIG. 9 is a flowchart of an imaging method of an imaging device according to some embodiments of the present invention.

### DESCRIPTION OF EMBODIMENTS

Specific implementation manners of the present invention will be described in the following. It should be noted that during the specific description of the implementation manners, it is impossible to describe all features of the actual implementation manners in detail in the present invention for the sake of brief description. It should be understood that in the actual implementation of any of the implementation manners, as in the process of any engineering project or design project, a variety of specific decisions are often made in order to achieve the developer's specific objectives and meet system-related or business-related restrictions, which will vary from one implementation manner to another. Moreover, it can also be understood that although the efforts made in such development process may be complex and lengthy, for those of ordinary skill in the art related to content disclosed in the present invention, some changes in design, manufacturing, production or the like based on the technical content disclosed in the present disclosure are only conventional technical means, and should not be construed as that the content of the present disclosure is insufficient.

Unless otherwise defined, the technical or scientific terms used in the claims and the description are as they are usually understood by those of ordinary skill in the art. “First,” “second” and similar words used in the present invention and the claims do not denote any order, quantity or importance, but are merely intended to distinguish between different constituents. “One,” “a(n)” and similar words are not meant to be limiting, but rather denote the presence of at least one. The word “include,” “comprise” or a similar word is intended to mean that an element or article that appears before “include” or “comprise” encompasses an element or article and equivalent elements that are listed after “include” or “comprise,” and does not exclude other elements or articles. The word “connect,” “connected” or a similar word is not limited to a physical or mechanical connection, and is not limited to a direct or indirect connection.

Although some embodiments of the present invention are presented in the particular context of human breast ultrasound, it should be understood that the present invention is applicable to ultrasound scanning of any externally accessible human or animal body part (for example, abdomen, legs, feet, arms, or neck), and is also applicable to other medical imaging devices (for example, X-ray scanning) with a similar mechanical structure. Moreover, although some embodiments of the present invention are presented in the particular context of mechanized scanning, it should be understood that the present invention is also applicable to a handheld scanning context.

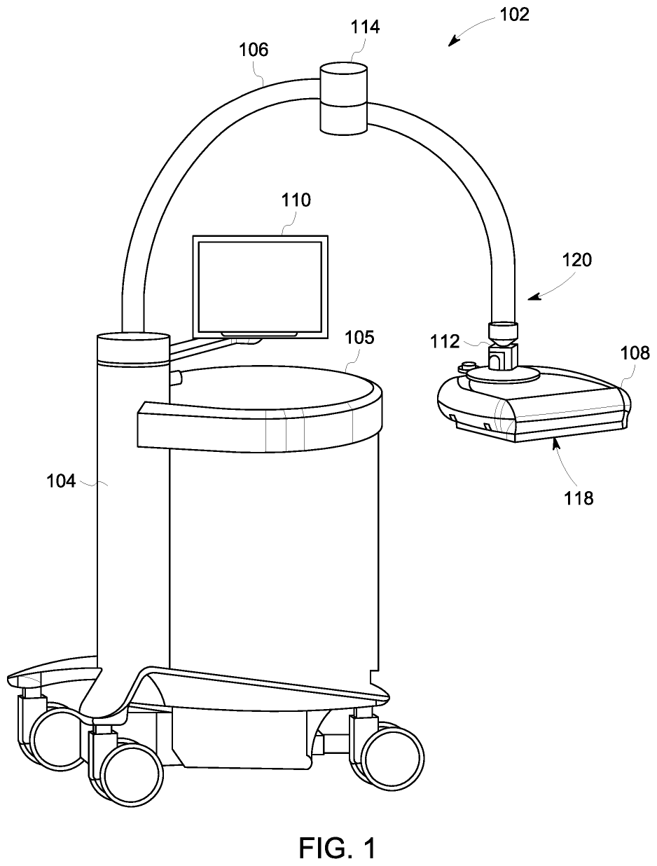

Referring to FIG. 1, it is a perspective view of a breast ultrasound scanning device 102 (hereinafter also referred to as a scanning device 102) according to some embodiments of an imaging device of the present invention. The scanning device 102 includes a frame 104, an ultrasonic processor housing 105 including an ultrasonic processor, an adjustable arm 106 including a hinge joint 114, an imaging assembly 108 connected to one end 120 of the adjustable arm 106 through a ball joint 112, and a display 110 connected to the frame 104. The imaging assembly 108 includes an ultrasonic transducer. The display 110 is connected to the frame 104 at a joining point where the adjustable arm 106 enters the frame 104. Since the display 110 is directly connected to the frame 104 rather than the adjustable arm 106, the display 110 does not affect the weight of the adjustable arm 106 and the balancing mechanism of the adjustable arm 106. In some embodiments, the display 110 may be rotated in a horizontal and transverse direction (for example, rotatable about a central axis of the frame 104), but not vertically. In some other embodiments, the display 110 may also be vertically movable.

It should be noted that FIG. 1 illustrates, as a reference, some configurations and relative positions of various components, but these configurations and relative positions are not limiting. For example, the position of the display 110 is not limiting; for example, the display 110 may be disposed on the ultrasonic processor housing 105, or may be freely disposed independently of the frame 104 or the housing 105. The shape of the adjustable arm 106 is not necessarily curved as in FIG. 1, the adjustable arm 106 may also have a polyline-shaped structure or even straight line shaped structure, and the adjustable arm 106 may also not include the hinge joint 114 but be integrally formed or have any other type of configuration without affecting the implementation of various embodiments of the present invention. In addition, the arrangement of the ball joint 112 is not limiting, and other types of connection may also be selected to connect the adjustable arm 106 and the imaging assembly 108. In some embodiments, the imaging assembly 108 includes a film 118 that is in a substantially tensioned state to be at least partially attached, for pressing the breast. The film 118 has a bottom surface for contacting the breast, and when the bottom surface is in contact with the breast, the transducer sweeps over a top surface of the film 118 to scan the breast. The film 118 may be a tensioned fabric sheet.

In some embodiments, the adjustable arm 106 is configured in a manner in which the imaging assembly 108 has a net downward weight of substantially zero or has a light net downward weight (for example, 1 to 2 Kg). With such net downward weight, the position of the imaging assembly 108 can be freely adjusted by a user and the imaging assembly 108 can remain stationary after the adjustment. In some other embodiments, after the adjustment is made such that the imaging assembly 108 is brought into contact with tissue to be scanned, the internal components of the scanning device 102 may be adjusted to apply a desired downward weight to press the breast and improve imaging quality. In some embodiments, the net downward weight may be in the range of 2 to 11 Kg. The adjustment of the weight of the imaging assembly 108 will be illustrated in detail below.

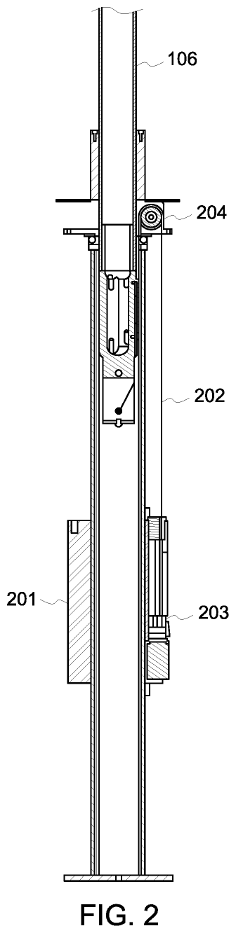

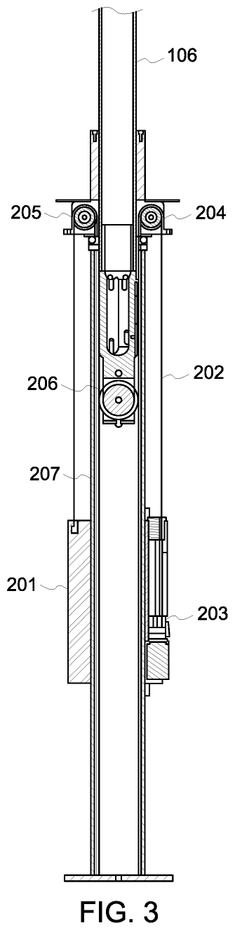

Referring to FIGS. 2 and 3, schematic views illustrating an adjusting structure of the adjustable arm 106 in some embodiments of the present invention are shown. One end of the adjustable arm 106 is connected to the imaging assembly 108 (the imaging assembly 108 is not shown in FIGS. 2 and 3), and the other end thereof is connected to a counterweight 201 through a cable 202. It should be noted that the meaning of connection through the cable 202 is not limited to a fixed connection between the adjustable arm 106 and/or counterweight 201 and the cable 202. Such a connection may also be in any other form, such as a slidable connection. The adjustable arm 106 and the counterweight 201 should be considered connected to each other as long as the cable 202 enables certain interaction force between the adjustable arm 106 and the counterweight 201. In addition, the description of one end and the other end does not limit the arrangement to two ends of the adjustable arm 106, but is used to distinguish two different arrangement positions, where the two positions may be two ends of the adjustable arm 106, or may be positions close to the ends. The counterweight 201 is further provided with an actuator 203, and one end of the cable 202 is connected to the actuator 203 so that the actuator 203 can drive the cable 202 to move. In some embodiments, the weight of the counterweight 201 is specially designed and is basically equal to the sum of the weight of the imaging assembly 108 and the weight of the adjustable arm 106. It is found in the present invention that such an arrangement ensures that the imaging assembly 108 is neutrally buoyant in a vertical direction; that is, the net downward weight thereof is basically zero, making it convenient for an operator to adjust the position thereof. Certainly, the weight configuration of the counterweight 201 is not limiting, and the weight of the counterweight 201 may also be greater or less than the sum of the weight of the imaging assembly 108 and the weight of the adjustable arm 106. The operator may also adjust the length of the cable 202 by controlling the actuator 203, thereby achieving the adjustment of the position of the adjustable arm 106. When the imaging assembly 108 is attached to the surface of tissue to be imaged, such adjustment will change the pressure applied by the imaging assembly 108 on the tissue to be imaged, thereby adjusting the effective downward weight of the imaging assembly 108. The imaging quality can be adjusted by adjusting such pressure changes. It can be seen that such arrangement simplifies the configuration of the counterweight 201, and the adjustment of the position of the imaging assembly 108 and adjustment of the pressure applied by the imaging assembly 108 on the tissue to be imaged can be achieved without any additional design.

Some embodiments of the present invention provide the connection mode between the adjustable arm 106 and the cable 202. Referring to FIG. 2, the cable 202 may be fixedly connected to the other end of the adjustable arm 106 and then connected to the counterweight 203 through a first pulley 204. Such arrangement enables a more secure connection between the adjustable arm 106 and the cable 202 and the problems such as cable fall-off during use do not easily occur. It should be noted that the first pulley 204 is not always required, and the cable 202 may also be guided by other structures. For example, a smooth column structure can also be used to guide the cable 202. In some embodiments, a plurality of first pulleys 204 may also be provided to enable more smooth guiding of the cable 202. The cable 202 may also be configured as a chain structure, and correspondingly, the first pulley 204 may be configured as a gear structure. Such configuration can avoid slipping. Referring to FIG. 3, another connection mode for the cable 202 and the adjustable arm 106 is provided. In some embodiments, a third pulley 206 is disposed at the bottom of the adjustable arm 106. One end of the cable 202 is connected to the actuator 203 on the counterweight 201, and the other end of the cable 202 is fixedly connected to the counterweight 201 through the first pulley 204, the third pulley 206 at the bottom of the adjustable arm 106, and a second pulley 205. Such arrangement achieves a slidable connection between the cable 202 and the bottom of the adjustable arm 106, and enables a smooth adjustment of the position of the adjustable arm 106. In some embodiments, a connecting line between the first pulley 204 and the second pulley 205 basically passes through a central position of the adjustable arm 106. Such arrangement ensures that the force applied by the counterweight 201 to the adjustable arm 106 through the cable 202 is approximately in the vertical direction, thereby enabling a smooth adjustment of the position of the adjustable arm 106.

In some embodiments, the frame 104 may further include a structure of guide rail 207. The guide rail 207 can be used for guiding the counterweight 201 and the adjustable arm 106, thereby enabling a smooth adjustment of the position of the adjustable arm 106. When the guide rail 207 is provided, the first pulley 204 and/or second pulley 205 described above may be disposed on the guide rail structure to facilitate fixed connection of the pulleys. It should be noted that the guide rail structure is not always required, and the pulley structure may also be fixedly disposed on the frame 104 rather than the guide rail 207. In some embodiments, the guide rail 207 may have a variety of shapes, for example, a hollow structure as shown in FIGS. 2 and FIG. 3. Such structural configuration enables the inner portion of the guide rail 207 to accommodate and make contact with at least part of the adjustable arm 106, and the outer portion of the guide rail 207 to make contact with at least part of the counterweight 201. Optionally, the adjustable arm 106 and the counterweight 201 are also provided with guide structures matching the guide rail. These guide structures will be discussed in detail below. In addition to the configuration shown in FIGS. 2 and 3, the guide rail 207 may also be configured as other structural types, such as a structure of a pillar type. The guide rail 207 of such structure allows part of the adjustable arm 106 to be disposed on one surface of the pillar of the guide rail 207, and at least part of the counterweight 201 to be disposed on the other surface of the pillar of the guide rail 207.

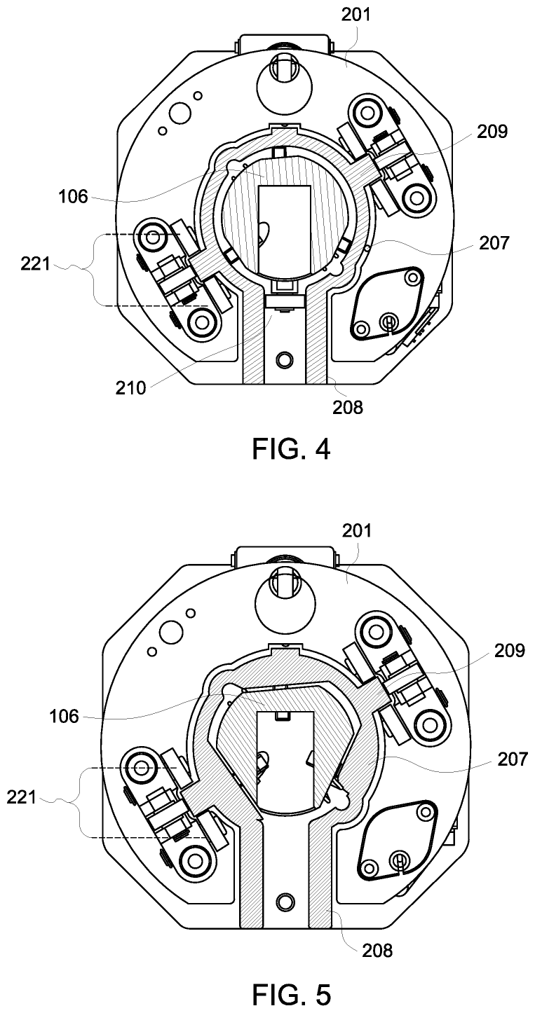

Referring to FIG. 4 and FIG. 5, the guiding structure of the guide rail 207 is illustrated in greater detail. Referring first to FIG. 4, a bottom view of the guide rail 207 according to some embodiments of the present invention is shown. In some embodiments, the guide rail 207 is a hollow cylindrical structure and provided with an outward protruding gap portion 208, and several protrusions 209 are disposed on an outer wall of the guide rail. Accordingly, the adjustable arm 106 is partially disposed in the guide rail 207, a position-limiting structure 210 is disposed on an outer side of the adjustable arm 106, and the position-limiting structure 210 is accommodated in the gap portion 208. In some embodiments, the position-limiting structure 210 may be a pulley or pulley block. Such arrangement ensures that the adjustable arm 106 does not have unexpected swinging during position adjustment, thereby facilitating position fixing by the operator. Under the teachings of the present invention, those skilled in the art could also configure the position-limiting structure 210 as any structure other than a pulley, such as any other type of protrusions. In addition to the position-limiting structure 209, a plurality of pulley structures may further be disposed on the outer wall of the adjustable arm 106 to enable a smooth movement in the guide rail 207. The specific structure of the adjustable arm 106 will be illustrated in detail below. In some embodiments, at least part of the counterweight 201 is connected to the outer side of the guide rail 207. In some embodiments, the counterweight 201 is configured as a ring-shaped structure surrounding the outer side of the guide rail 207 and provided with an opening portion for accommodating the aforementioned outward protruding gap portion 208. In some embodiments, the counterweight 201 may further be provided with several pulley block structures 221 used for engagement with the several protrusions 209 on the outer wall of the guide rail. Such arrangement enables the counterweight 201 to slide more smoothly along the guide rail 207 without rotation, thereby avoiding knotting or tangling of the cable 202 during the movement of the counterweight 201. In some embodiments, each pulley block structure 221 is configured to be formed of three pulleys as shown in FIG. 4, and the three pulleys respectively make contact with three surfaces of the protrusion 209. Such arrangement ensures that the counterweight 201 moves smoothly relative to the guide rail 207, because during the movement of the counterweight 201, no matter to which position the counterweight 201 shifts, the pulleys in the pulley block structure 221 can be slidably connected to the protrusion 209 of the guide rail 207. Such arrangement also limits the position of the counterweight 201 to avoid its rotation around the guide rail. Some more detailed embodiments about the counterweight 201 are discussed in FIG. 7 and below. It should be noted that FIG. 4 merely shows the pulley block structures 211 at the bottom of the counterweight 201. In the actual configuration, more pulley block structures 221 may be provided, for example, also at the top of the counterweight 201. In addition, the pulley block structure 221 may also not be provided.

Still referring to FIG. 5, a guiding mode of the guide rail 207 in some other embodiments of the present invention is shown. In some embodiments, the cross section of the guide rail 207 may be a non-circular structure. FIG. 5 shows an embodiment of a non-circular structure, where a portion of the guide rail 207 matching the adjustable arm 106 is configured as a triangular structure. In some embodiments, the triangular guide rail 207 may be an equilateral triangle. Such configuration will facilitate installation of the counterweight 201 and the adjustable arm 106. The guide rail 207 of the non-circular structure and the counterweight 201 and the adjustable arm 106 that match the guide rail 207 eliminate the need of providing the position-limiting structure 210 as in FIG. 4, because such structure itself has a position-limiting function so that the counterweight 201 and the adjustable arm 106 do not freely rotate. Certainly, the cross section of the outer contour of the guide rail 207 may also be configured as a triangular structure, and accordingly, the protrusions 209 and the pulley block structures 221 do not need to be provided. It should be noted that under the teachings of the technical solution of the present invention, those skilled in the art could also provide the protrusions 209, the position-limiting structure 210, and the pulley block structures 221 to further improve the position-limiting function of the guide rail 207. That is, the implementations in FIG. 4 and FIG. 5 are not mutually exclusive, and can be freely combined according to the teachings of the present invention. In addition, FIG. 5 merely shows some implementations of the non-circular guide rail 207. In addition to the triangular structure, any other non-circular structures may also be used, such as a rectangle, an oval, and other irregular polygons.

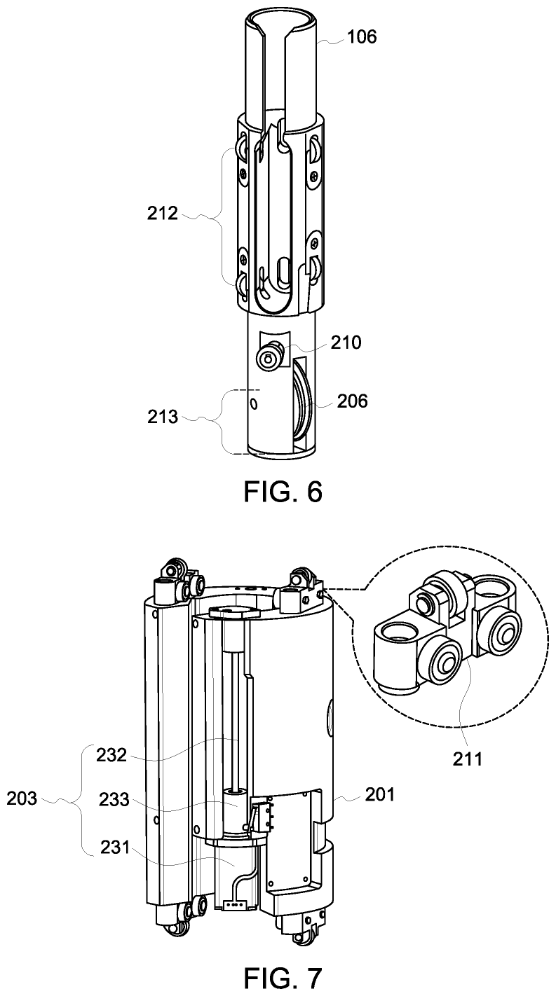

Referring to FIG. 1 and FIG. 6, the structure of a portion of the adjustable arm 106 in contact with the guide rail 207 is illustrated in further detail. Referring to FIG. 6, a schematic structural view of a portion of the adjustable arm in contact with the guide rail according to some embodiments of the present invention is shown. In some embodiments, one end of the adjustable arm 106 includes the third pulley 206 disposed at the bottom of the adjustable arm 106 and the position-limiting structure 210 disposed on the outer side of the adjustable arm 106. Additionally, in some embodiments, a plurality of guide pulleys 212 are further disposed on the outer side of the adjustable arm 106. It can be seen with reference to FIG. 1 and FIG. 6 that such arrangement of the guide pulleys 212 enables less friction produced when the adjustable arm 106 is in contact with the guide rail 207, making it convenient for the operator to freely adjust the height of the adjustable arm 106 during use. In some embodiments, a blocking plate 213 is disposed on the outer side and the bottom of the third pulley 206, and the blocking plate 213 at least partially surrounds the third pulley 206. During device assembly, the cable 202 passes through the gap between the blocking plate 213 and the third pulley 206 to make contact with the third pulley 206. In some embodiments, the spacing between the blocking plate 213 and the third pulley 206 is set to be less than the diameter of the cable 202. Such configuration can achieve at least the following technical effect: the cable 202 will be better fixed after being disposed between the blocking plate 213 and the third pulley 206, and does not easily fall off or get stuck during use.

In some embodiments, the portion of the adjustable arm 106 in contact with the guide rail 207 is designed to be detachable from the remaining portion of the adjustable arm 106 (including the portion connected to the imaging assembly 108). Such design allows the two portions of the adjustable arm 206 to be connected to each other by means of fastening, thereby enabling a more convenient installation. More preferably, the two portions are rotatably connected. It is found in the present invention that such configuration also has obvious advantages in other aspects: in one aspect, because of the position-limiting structure or the design of a non-circular structure, it is ensured that the portion of the adjustable arm 106 in contact with the guide rail 207 does not rotate in the horizontal plane direction during lifting of the adjustable arm 106, thereby avoiding tangling and knotting of the cable 202 and other power supply lines, and improving instrument stability; in another aspect, the remaining portion of the adjustable arm 106 is rotatably connected, which ensures that the imaging assembly 108 has the function of rotating in the horizontal plane, and thus the position can be adjusted more easily at several angles during use to approach the tissue to be imaged. It should be noted that such detachable configuration is not necessarily required, and the adjustable arm 206 may also be configured in an integral forming design. In addition, the adjustable arm 206 does not need to rely on the guide rail 207 in some embodiments as described above in the present invention.

Referring to FIG. 7, a schematic structural view of the counterweight 201 in some embodiments of the present invention is shown. In some embodiments, pulley block structures 221 are disposed at many positions including the upper part and lower part of the counterweight 201, and each pulley block structure 221 may be provided with several fixed pulleys, such as three fixed pulleys as shown in FIG. 7. Such arrangement ensures that the friction is reduced as much as possible after the counterweight 201 makes contact with the several protrusions 209 on the outer wall of the guide rail 207. In some embodiments, in order to facilitate the processing and assembly of the pulley block structure 221, the pulley block structure 221 and the counterweight 201 may be configured to be detachably connected. For example, screw holes are provided on the pulley block structure 221 and the counterweight 201, and the two are connected by a bolt. It should be noted that the bolt connection is not the only connection mode for two components, and other detachable connection modes are also possible. In addition, the pulley block structure 211 may also be designed to be integrally formed with the counterweight. Each protrusion 209 on the outer wall of the guide rail 207 may correspond to two pulley block structures 221, so that the counterweight 201 can move more smoothly along the guide rail. The two pulley block structures 221 may be disposed at upper and lower ends of the counterweight. It should be noted that the number and positions of the pulley block structures 221 may also be freely adjusted.

Still referring to FIG. 7, a schematic structural view of the actuator 203 in some embodiments of the present invention is shown. In some embodiments, the actuator 203 is configured in such a manner that a lead screw motor 231 operates in coordination with a sliding block 233. For example, the lead screw motor 231 may be disposed at the bottom of the counterweight, a screw rod 232 extends out from the bottom up, the screw rod 232 is provided with threads, and the sliding block 233 is provided with internal threads and is sleeved on the screw rod 232. With such configuration, during rotation of the lead screw motor 231, the sliding block 233 can be driven to move along with the rotation by means of the screw rod 232, and the lead screw motor 231 can rotate forward or backward so that the sliding block 233 moves up or down (or possibly moves down or up). In some embodiments, one end of the cable 202 (the cable 202 is not shown in FIG. 7) is connected to the sliding block 233, so that the cable 202 can move when driven by the sliding block 233. As described above, the cable 202 connects the adjustable arm 106 and the counterweight 201, and the cable 202 moves when driven by the sliding block 233, which will result in fine adjustment of the position of the adjustable arm 106 in a vertical height direction. Such fine adjustment also causes the movement of the imaging assembly 108 at the other end of the adjustable arm 106, thereby adjusting the effective weight applied by the imaging assembly 108 to the tissue to be imaged. Such configuration can conveniently adjust the effective weight to adjust imaging quality, and can also reduce to a great extent the structural complexity of an effective weight adjustment device due to the compact design. It should be noted that FIG. 7 shows some embodiments of the structure of the actuator 203, but the actuator 203 may have other configurations. For example, the lead screw motor 231 may be disposed at any position in the actuator 203, such as on the upper part or middle part of the counterweight 201 or at any other position. In addition, the actuator 203 does not necessarily employ the mode of driving by the lead screw motor 231. Under the teachings of the present invention, any other type of structure of the actuator 203 is possible. For example, a motor with a gear is provided and used with a rack for driving to achieve movement adjustment of the cable 202; or a motor with a driving belt structure is provided to drive the cable 202 to move. To sum up, the present invention has described in detail the principles of the actuator 203 driving the cable 202 to move and the prominent beneficial effects over the prior art, and under the teachings of the present invention, those skilled in the art could select other possible types of the actuator 203 according to the prior art.

In some embodiments, the counterweight 201 is further provided with a locking device 300, and the locking device 300 can be used to lock the counterweight 201 to achieve position fixing after the counterweight 201 is adjusted by the operator to any desired position. The function of the locking device 300 itself is to fix the position of the counterweight 201. Therefore, any devices in the prior art capable of fixing the position of a counterweight and having the locking function can be used for locking and position fixing for the counterweight 201 in the present invention. These devices will not be enumerated in the present invention, and only several examples of the locking device 300 will be provided below. It should be noted that the following are only several examples of the locking device 300, not limitations to the locking device 300 that can be used by the present imaging device. Furthermore, although the structural configuration of the locking device 300 may be freely configured in the prior art, the application of the locking device 300 achieves obviously better effects since in the present invention, the interaction between the locking device 300 and other components of the imaging device, especially the interaction between the locking device 300 and the aforementioned actuator 203, is used to achieve adjustment of the net effective weight of the tissue to be imaged.

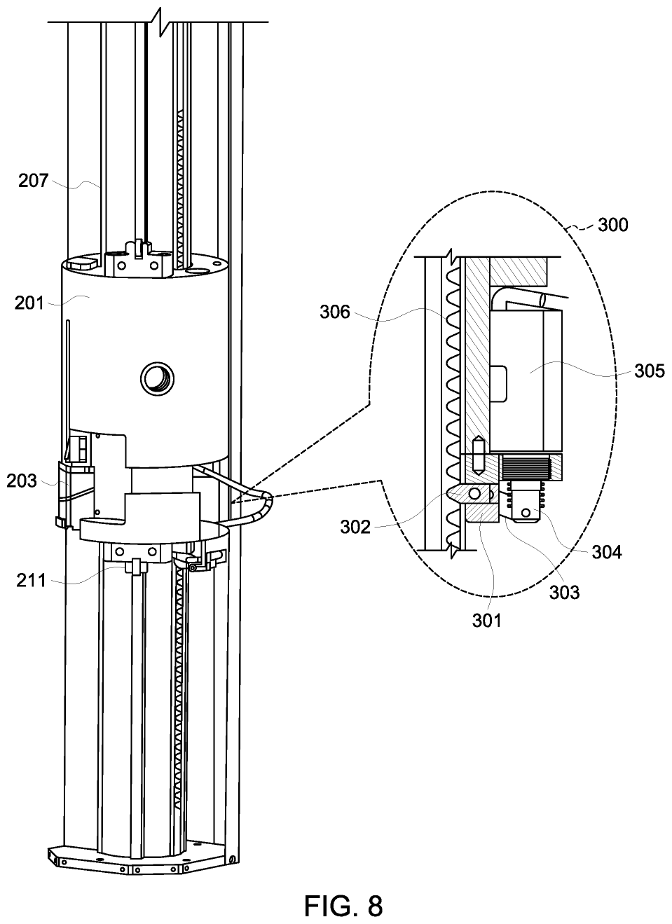

Referring to FIG. 8, the structure of the locking device 300 in some embodiments of the present invention is shown. The locking device 300 is used for fixing the position of the counterweight. The locking device 300 includes a through-hole 301, a pin 302, a first connecting rod 303, a pressing rod 304, a second actuator 305, and a recess 306.

In some embodiments, the through-hole 301 is provided in the counterweight 201 and close to the guide rail 207, thereby facilitating the engagement of the pin 302 with the recess 305 provided in the guide rail 207 during locking. It should be noted that the term “being close” means that the through-hole 301 may be provided in a manner to either keep a distance from the guide rail 207, or be attached to the guide rail 207. The through-hole 301 can be arranged in a variety of ways. For example, a protruding structure is disposed on the counterweight 201, and the through-hole 301 is provided in the protruding structure. In some embodiments, the protruding structure may be integrally formed with the counterweight 201, or may be detachably connected to the counterweight 201, and in both cases the protruding structure is considered as part of the counterweight 201. Other configurations are also possible, for example, a configuration in which a protruding structure is not provided, and instead, the through-hole 301 is provided directly in the main body of the counterweight 201.

In some embodiments, the pin 302 is at least partially disposed in the through-hole 301. The phrase “being at least partially disposed” means that the pin 302 may partially pass through the through-hole 301 as shown in FIG. 8. A longer structure of the through-hole 301 may also be provided so that the pin 302 is mostly or even entirely disposed in the through-hole 301. Such configuration will enable the pin 302 to be better guided by the through-hole during movement.

In some embodiments, one end of the first connecting rod 303 is movably connected to the pin 302. The first connecting rod 303 may have a variety of shapes, including, but not limited to, a rod shape, a sheet shape, and a dumbbell shape, as long as it is a structure having a connection function. The movable connection between the first connecting rod 303 and the pin 302 has a variety of modes, which may be a connection using a pin shaft and a pin hole matching each other or a connection using a hook-shaped structure and/or a ring-shaped structure, which will not be enumerated herein. The connecting point at which the pin 302 is connected to the first connecting rod 303 may be located at an end of the pin 302, or at a middle position of the pin 302, or other positions. The aforementioned configuration enables the first connecting rod 303 to drive the pin 302 to move.

In some embodiments, the pressing rod 304 is movably connected to the other end of the first connecting rod 303. As described above, the movable connection has a variety of modes, which may be a connection using a pin shaft and a pin hole matching each other or a connection using a hook-shaped structure and/or ring-shaped structure, which will not be enumerated herein. It should be noted that a connection between two components does not necessarily mean a physically or mechanically direct connection. The other end of the first connecting rod 303 is connected to the pressing rod 304 through any other driving structure, which is also a connection mode included in the present invention, for example, connected to the pressing rod 304 through a second connecting rod 307. Furthermore, the expression of the other end of the first connecting rod 303 is used for differentiation from the aforementioned one end of the first connecting rod 303, and does not mean that the other end is necessarily located at an end of the first connecting rod 303, which may also be a position close to an end, even a position close to the middle part, or other positions. By means of such configuration, the pressing rod 304 can drive the first connecting rod 303 to move and then drive the pin 302 to move in certain directions.

In some embodiments, although the pressing rod 304 can move by manual adjustment, for the purpose of automation, the second actuator 305 may be provided to drive the pressing rod to move. The mode of actuation between the second actuator 305 and the pressing rod 304 may be in any mode well known to those skilled in the art, for example, a mode using screw rod/sliding block driving, a mode using electromagnet/magnet interaction, a mode using gear/rack driving, or a mode using motor/driving belt driving. FIG. 8 merely exemplarily shows the electromagnet/magnet actuation mode, in which the second actuator 305 is selected to be an electromagnetic actuator 305, the electromagnet 305 can generate a magnetic field in a power-on state, and the magnetic field disappears in a power-off state. Accordingly, the pressing rod 304 includes at least a material attractable by the electromagnetic actuator 305, for example, a magnet, iron, stainless steel or other structures attractable by the electromagnet. The electromagnet actuator 305 will attract and move the pressing rod, thereby driving the pin 302 to move by means of the first connecting rod 303. Additionally, the electromagnetic actuator 305 can also drive the pressing rod 304 to move by a repulsive force. For example, a portion of the pressing rod corresponding to the electromagnetic actuator 305 is configured to have the same magnetic pole as that of the electromagnetic actuator 305, so that such a repulsive force can also achieve adjustment of the movement of the pressing rod 304.

In some embodiments, at least one recess 306 is provided on the frame 104 and used for achieving engagement with the pin 302 in a locked state. The recess 306 may have any shape, such as a circle, a square, a triangle, or a hexagon, as long as the function of accommodating part of the pin can be achieved, so that the recess can at least partially match the pin during locking, thereby achieving effective locking of the counterweight 201 to the frame 104. In some embodiments, one recess 306 may be provided. In some embodiments, a plurality of recesses 306 may be provided, so as to achieve locking of the counterweight 201 to the frame 104 at a plurality of positions. In some embodiments, the plurality of recesses are configured to be arranged in a sliding direction of the counterweight 201 relative to the frame 104. The spacing between the plurality of recesses may be freely configured. For example, if the counterweight 201 needs to be locked to the frame 104 at a plurality of positions in the relative sliding direction, the spacing between the plurality of recesses may be appropriately decreased, thereby improving the precision of locking and position fixing; if high precision is not required, the spacing between the plurality of recesses may be appropriately increased, which facilitates reducing the processing difficulty for the recesses. To sum up, under the aforementioned teachings of the present invention, the number and positions of the recesses 306 may be freely adjusted according to actual needs to achieve the aforementioned different technical effects. It should be noted that although a configuration of a locking mechanism is described in detail above, under the teachings of the present invention, such configuration of the locking mechanism is not limiting and may be freely selected as long as the counterweight can be locked to satisfy at least some functions in the present invention.

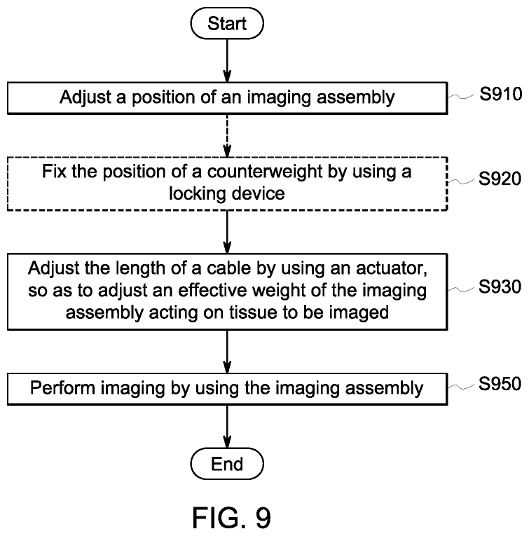

Referring to FIG. 9, a flowchart of an imaging method in some embodiments of the present invention is disclosed. The imaging method may be implemented by the imaging device in any of the embodiments described above. The imaging method of the imaging device in any of the embodiments of the present invention is now further illustrated.

At S910, a position of the imaging assembly 108 is adjusted so that the imaging assembly is close to a surface of tissue to be imaged. According to the above disclosure, in some embodiments, the weight of the counterweight 201 is specially designed and is approximately equal to the sum of the weights of the adjustable arm 106 and the imaging assembly 108. In this case, an operator can easily adjust the position of the imaging assembly 108. Since such configuration ensures that the imaging assembly 108 is substantially neutrally buoyant, only a small upward or downward force is needed to apply to the imaging assembly 108 so as to adjust the movement thereof in the vertical direction, so that the imaging assembly 108 can be close to the surface of the tissue to be imaged to prepare for the subsequent imaging. The force applied by the imaging assembly 108 to the tissue to be imaged has an important impact on the imaging quality; an excessively large or small force is disadvantageous to improving the imaging quality, and therefore needs to be adjusted before imaging.

At S930, the length of the cable 202 is adjusted by using the actuator 203. Specifically, after the imaging assembly 108 gets close to the surface of the tissue to be imaged, the actuator 203 drives the cable 202 to extend, so that the pulling force of the counterweight 201 on the imaging assembly 108 through the cable decreases. The decrease of the pulling force will further result in an increase of the effective downward weight of the imaging assembly 108 on the tissue to be imaged, so that the effective weight of the imaging assembly 108 on the tissue to be imaged can be adjusted increasingly. On the contrary, when the pressure of the imaging assembly 108 on the tissue to be imaged is excessively large, the actuator 203 may be adjusted to drive the cable 202 to retract. In this way, the pulling force of the counterweight 201 on the imaging assembly 108 through the cable increases. The increase of the pulling force will further result in a decrease of the effective downward weight of the imaging assembly 108 on the tissue to be imaged, so that the effective weight of the imaging assembly 108 on the tissue to be imaged can be adjusted decreasingly. The pressure of the imaging assembly 108 is adjusted through the counterweight 201 in combination with the cable 202, rather than through a direct gear or other direct mechanical driving. Such pressure adjustment is gentle, and even if a failure occurs that a mechanical device gets out of control or gets stuck, no serious damage will be caused to the tissue to be imaged, and the imaging assembly 108 can be easily removed from the tissue to be imaged. It should be noted that although S930 can be implemented by manually or directly controlling the actuator, for the purpose of convenience to the operator, the instruction is, more preferably, executed by a program to automatically adjust the actuator according to an input instruction of the operator, for example, by controlling a corresponding button on the imaging assembly 108.

At S950, imaging is performed by using the imaging assembly 108. The method for imaging using the imaging assembly 108 may be freely selected, and may be determined according to the specific imaging device. In some embodiments, the imaging device may be configured as the breast ultrasound scanning device 102 as described above. In this case, the imaging assembly 108 may be an assembly including an ultrasonic transducer to perform ultrasound scanning. In addition, other imaging types are also possible, for example, imaging means such as ultrasound scanning on other tissue of the human body or animal body or even X-rays, which will not be described herein again.

It should be noted that the imaging method of FIG. 9 merely shows critical steps in some embodiments of the present invention, which, however, is not a limitation of these methods being constituted solely by these steps. For example, in order to make it easier to adjust the effective weight of the imaging assembly 108, step S20 of locking the counterweight by using the locking device 300 described above may also be included. The configuration of the locking device 300 may be seen in some embodiments above, or may be freely selected in the prior art. Optionally, step S20 may be selected to be after step S10, namely, after adjusting a position of the imaging assembly 108 so that the imaging assembly is close to a surface of tissue to be imaged. After the approximate position of the imaging assembly 108 is determined, the position of the counterweight 201 is fixed by using the locking device 300 so that the subsequent adjustment of the effective weight of the imaging assembly 108 can be more efficient. The counterweight 201 is locked so that when the operator controls the actuator 203 to drive the cable 202 to extend or retract, the position of the counterweight 201 will not change according thereto. At this point, the change in the length of the cable 202 will directly affect the effective weight applied by the imaging assembly 108 to the tissue to be imaged.

The purpose of providing the above specific embodiments is to facilitate understanding of the content disclosed in the present invention more thoroughly and comprehensively, but the present invention is not limited to these specific embodiments. Those skilled in the art should understand that various modifications, equivalent replacements, and changes can also be made to the present invention and should be included in the scope of protection of the present invention as long as these changes do not depart from the spirit of the present invention.

What is claimed is:

1. An imaging device, comprising:
an adjustable arm;
an imaging assembly connected to one end of the adjustable arm; and
a counterweight connected to the other end of the adjustable arm through a cable, wherein the counterweight is provided with an actuator, and the actuator is connected to one end of the cable and is capable of driving the cable to move.

2. The device according to claim 1, further comprising a frame, wherein the frame comprises a guide rail, and the guide rail is configured to guide the counterweight and/or the adjustable arm during movement.

3. The device according to claim 2, wherein the guide rail is a hollow structure having an inner portion in contact with the adjustable arm and an outer portion in contact with the counterweight.

4. The device according to claim 3, wherein the adjustable arm comprises a position-limiting structure for limiting a position of the adjustable arm during movement.

5. The device according to claim 3, wherein portions of the guide rail in contact with the adjustable arm are all non-cylindrical structures.

6. The device according to claim 3, wherein a portion of the adjustable arm in contact with the guide rail is rotatably connected to the remaining portion of the adjustable arm.

7. The device according to claim 1, wherein the other end of the cable is fixedly connected to the adjustable arm.

8. The device according to claim 1, wherein one end of the cable is slidably connected to a bottom of the adjustable arm, and the other end thereof is connected to the counterweight.

9. The device according to claim 8, wherein the bottom of the adjustable arm is provided with a pulley and a blocking plate at least partially surrounding the pulley, a spacing between the pulley and the blocking plate is less than the diameter of the cable, and the cable is slidably connected to the bottom of the adjustable arm through the pulley.

10. The device according to claim 1, wherein the actuator comprises a lead screw motor and a sliding block connected to the lead screw motor, and the sliding block is connected to one end of the cable.

11. The device according to claim 1, further comprising a frame and a locking device, wherein the locking device is configured to fix a position of the counterweight.

12. The device according to claim 11, wherein the locking device comprises:
a through-hole, the through-hole being provided in the counterweight at a position close to the frame;
a pin, the pin being at least partially disposed in the through-hole;
a first connecting rod and a pressing rod, one end of the first connecting rod being movably connected to the pin, and the other end thereof being movably connected to the pressing rod;
a second actuator, the second actuator driving the pressing rod to move; and
at least one recess, the recess being provided on the frame and matching the pin.

13. The device according to claim 1, wherein the imaging assembly comprises an ultrasonic transducer.

14. A method for imaging using the imaging device according to claim 1, comprising:
adjusting a position of an imaging assembly;
adjusting the length of a cable by using an actuator, so as to adjust an effective weight of the imaging assembly acting on tissue to be imaged; and
performing imaging by using the imaging assembly.

15. The method according to claim 14, further comprising fixing a position of a counterweight by using a locking device.
